(19) United States
(12) Patent Application Publication
(10) Pub. No.: US 2013/0046148 A1
(43) Pub. Date: Feb. 21, 2013

(54) Three Dimensional Penetrating Optical-Electrical Neural Interface for Selective Stimulation and Recording

(71) Applicants: Prashant Tathireddy, Salt Lake City, UT (US); Loren Rieth, Salt lake City, UT (US); Gregory Arthur Clark, Salt Lake City, UT (US); Richard A. Normann, Park City, UT (US); Florian Solzbacher, Salt Lake City, UT (US); Steven Blair, Salt Lake City, UT (US)

(72) Inventors: Prashant Tathireddy, Salt Lake City, UT (US); Loren Rieth, Salt lake City, UT (US); Gregory Arthur Clark, Salt Lake City, UT (US); Richard A. Normann, Park City, UT (US); Florian Solzbacher, Salt Lake City, UT (US); Steven Blair, Salt Lake City, UT (US)

(21) Appl. No.: 13/508,969

(22) PCT Filed: Nov. 9, 2010
(86) PCT No.: PCT/US2010/056050

§ 371 Date: Oct. 9, 2012

Related U.S. Application Data
(60) Provisional application No. 61/259,524, filed on Nov. 9, 2009.

(51) Int. Cl. A61N 5/06; A61B 5/05

(52) U.S. Cl. 600/300; 607/88

(57) ABSTRACT

A hybrid optical-electrical neural interface is disclosed and described. The neural interface can include an array (100) having a plurality of micro-optrodes (HO). The micro-optrodes (110) are capable of optical and optionally electrical stimulation and recording, allowing bidirectional, multi-modal communication with neural tissue. At least a portion of the plurality of micro-optrodes (110) are independently optically addressable and include an optical waveguide along each micro-optrode (HO). Combining optical stimulation with electrical recording can allow artifact-free recording from nearby electrodes and in some cases even the same electrode, which is difficult to achieve with combined electrical recording and stimulation. The optical waveguide is configured to direct light towards a distal end (125) of the micro-optrode, allowing focal stimulation and recording. Penetrating micro-optrodes (110) can allow access to deep tissue, while non-penetrating micro-optrodes can be used for extraneural stimulation.

### RELATED APPLICATIONS

This application claims the benefit of U.S. Patent Application Ser. No. 61/259,524, filed Nov. 9, 2009, the entire contents of which are incorporated herein by reference.

### BACKGROUND

Various neural interface devices are under development for numerous applications involving restoration of lost function due to traumatic injury or neurological disease. Often the neural interface approaches place sensors or electrodes in a specific area of the brain or peripheral nerve such that a stimulus or electrical signal sent to the electrode can produce a response, such as a movement of a particular muscle group and/or sensations. For example, the electrodes can enable thought control of voluntary movement of body parts.

In application, the electrodes can be used to control affected paralyzed regions, artificial limbs or prosthetic devices, as well as a number of other devices such as computers, robots, and the like. Paralysis, loss of limbs, and various other afflictions can result in a reduced quality of life. However, neural interfaces can assist an afflicted person with regaining functionality and with improving the quality of life.

Some neural interfaces activate a large group of neurons. However, such an approach has limited application due to the inability to selectively activate specific neurons. Also, some approaches result in significant damage to nerve tissues and can limit the usefulness of the neural interface. A neural interface system which enables selective activation of neurons and reduces damage to nerve tissue would further the development of a useful neural interface device and further enhance the usefulness of neural interface devices to an afflicted person.

### BRIEF DESCRIPTION OF THE DRAWINGS

FIG. 1 illustrates a 10×10 array of micro-optrodes separated by glass at the base in accordance with an embodiment of the present technology.

FIG. 2 shows several optrodes in a micro-optrode array coated with optical-guide material except at the tips in accordance with an embodiment of the present technology.

FIG. 3 is an expanded view of one micro-optrode from FIG. 2.

FIG. 4 is a side cross-section view of an optical neural interface using a solid shaft as a waveguide for transport of light in accordance with an embodiment of the present technology.

FIG. 5 is a side cross-section view of an optical neural interface using microchannels as a waveguide for transport of light in accordance with an embodiment of the present technology.

FIG. 6 is a partial side view of an optical neural interface base showing independently optically addressable optical waveguides in accordance with an embodiment of the present technology.

FIG. 7 is a partial side view of an optical neural interface base showing a base light source for multiple optical waveguides in accordance with an embodiment of the present technology.

FIGS. 8A-8F are schematic diagrams illustrating one approach for manufacturing optical neural micro-optrodes having microchannels therein in accordance with embodiments of the present technology.

FIG. 9 is a side cross-section view of an optical neural interface using Fresnel lenses to focus an optical beam through a micro-optrode in accordance with an embodiment of the present technology.

FIGS. 10A-10B are side cross-section views of optical neural interfaces having differing height optrodes in accordance with embodiments of the present technology.

FIG. 11 is an electron microscope image of a micro-optrode array, wherein rows of micro-optrodes are formed at different heights in accordance with an embodiment of the present technology.

FIGS. 12A-12B illustrate placement of multiple optical neural interfaces on a nerve or nerve bundle in accordance with embodiments of the present technology.

FIGS. 13-14 illustrate graphs showing the performance of various optical neural interfaces in accordance with embodiments of the present technology.

FIGS. 15-16 are flow diagrams of methods for optically stimulating neurons in accordance with an embodiment of the present technology.

These drawings are provided for illustration purposes only such that alterations in relative dimensions, contours, and designs can be made consistent with the following description.

### DETAILED DESCRIPTION

Although these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

Definitions

In describing and claiming the present invention, the following terminology will be used.

The singular forms “a,” “an,” and “the” include plural referents unless the context clearly dictates otherwise. Thus, for example, reference to “a coating” includes reference to one or more of such coatings and reference to “stimulating” refers to one or more such steps for accomplishing the desired stimulus.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, the term “electrode” or “microelectrode” can refer to an electrical conductor or an optrode. An “optrode”, as used herein, is an optical counterpart of an electrical electrode. For example, both an electrode and an optrode can be used to provide a signal or stimulus to biological tissue. The terms electrode or microelectrode as used herein encompass both electrical and optical electrodes unless otherwise specified. Usage of the terms optrode or micro-optrode refer to an electrode which includes at least an optical conductor (e.g., waveguide, optical fiber, lens, etc.), but which does not necessarily include an electrical electrode. However, as will be appreciated from the following discussion, electrical and optical electrodes can be formed and used together (e.g. an electro-optrode) in a same device in accordance with embodiments of the present technology. Thus, in some cases the electrodes can include an optical waveguide and an electrically conductive pathway along the same electrode body. Thus, micro-optrodes can be both optically and electrically addressable. Alternatively, optrodes can form a portion of the while a separate portion of the microelectrodes are electrically conductive. Such mixed arrays can have optrodes and electrodes in segregated regions or intermixed.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

Optical Neural Interface

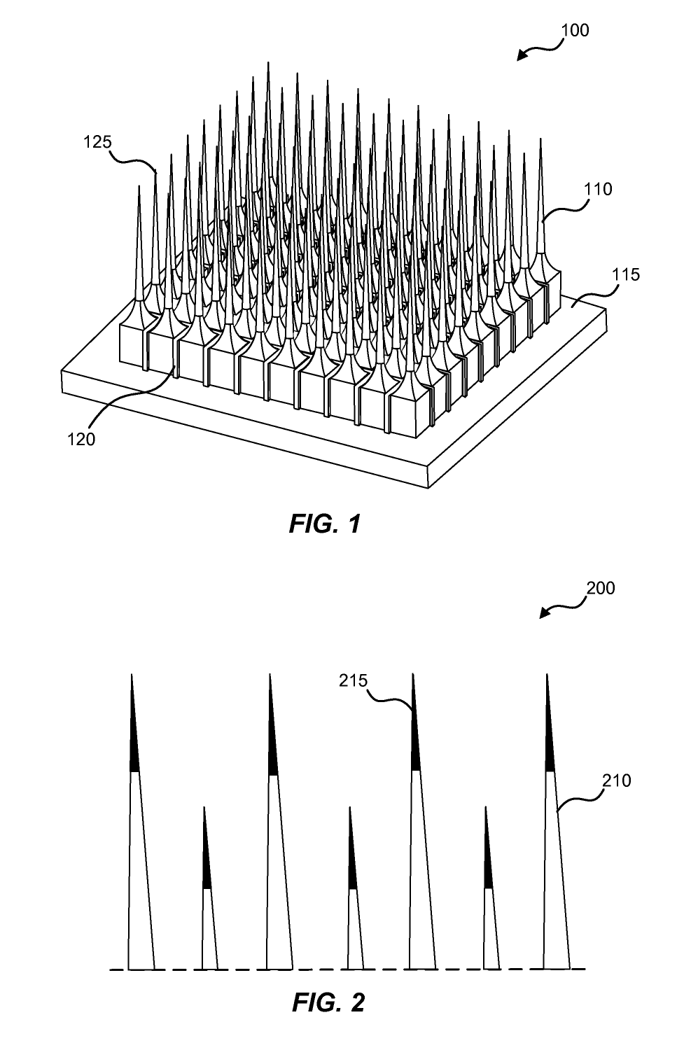

An optical neural interface can allow the presentation of a complex spatial temporal pattern of optical stimulation at specified depths of neural tissue. Such a device can be capable of guiding light to the tips of a microelectrode array. Any suitable microelectrode or micro-optrode array design can be used. The array 100 shown in FIG. 1 can be a Utah Optrode Array (UOA). Alternately, the array can be a Utah electrode array (UEA) which is described more fully in U.S. Pat. No. 5,215,088 and U.S. Patent Application Publication No. 20080138583, filed May 29, 2007, and which are each incorporated herein by reference. Other microelectrodes arrays can also be used in connection with the optical waveguide features described below. Non-limiting examples of arrays include the UEA; the Utah Slanted Electrode Array (USEA), which has electrodes of various lengths; planar arrays; the Utah Slanted Optrode Array (USOA), which has optrodes of various lengths; planar arrays and other penetrating microelectrode and/or micro-optrode arrays. The arrays can be secured on a common substrate 115 or base. Optionally, individual electrodes 110 or optrodes in the array can be independent of others. For example, an insulating material 120, such as a dielectric, can be filled in between the electrodes.

The plurality of microelectrodes can have tips 125 oriented in a common plane such as a traditional flat UEA, UOA, USEA or USOA. Alternatively, the tips can define a non-planar surface such as a contoured profile or varying heights across the array. Frequently, the entire plurality of microelectrodes are electrically isolated from one another via an insulating material such that they are independently addressable. However, in some cases a portion of the electrodes can be used as a ground or otherwise made dependent on other electrodes, e.g. as one or more reference electrodes. Resolution and performance can generally be improved by providing increased numbers of independently addressable microelectrodes. Typical electrode arrays can include 100 electrodes or more, such that implementation of the optical aspects presented below can allow a large number of independent stimulation and recording sites.

An optical neural interface can include an optical waveguide along at least one microelectrode so as to direct light towards a distal end of the microelectrode. In one alternative, a microelectrode array can have a plurality of microelectrodes secured in a common unit. Optionally, at least a first portion of the plurality of microelectrodes can be independently electrically addressable and at least a second portion of the plurality of microelectrodes includes an optical waveguide along the microelectrode. The first portion and the second portion of microelectrodes can, in some cases, include the same microelectrodes. However, in some aspects, the first portion and the second portion of microelectrodes can have some or no microelectrodes in common among their respective pluralities.

The optical waveguide can be formed in any profile that allows light to be transferred from the microelectrode near a distal end of the microelectrode. The light can be transferred sufficiently close to the tip to affect stimulation of a neuron in the vicinity of the tip. Generally, the optical waveguide can be formed of material which allows light of a given wavelength to travel its length. Various materials can be chosen and matched to a given wavelength. The optical waveguide can be formed of a material selected from the group consisting of silicon, silicon dioxide, silicon oxide, silica, acrylic, polystyrene, and combinations thereof. Even a 2-μm thick silicon oxide layer can act as a good optical wave guide for visible light. Specific choice of material can depend on the wavelength of light used, e.g. near-infrared, mid-infrared, visible, etc. Furthermore, suitable cladding material can be part of the optical waveguide to reduce transmission losses. For example, the cladding can be deposited along most of the length of the electrodes to prevent unwanted light escape by confining light along the optical waveguide, except at the tips, where light can be intentionally emitted. Metal films can be deposited on both curved surfaces (inner and outer surfaces) of the wave guide material to create good total-internal reflection and reduce light ‘escape’ during transfer to the tip. This technique can be used to transport light of practically any wavelength as long as the correct wave-guide material is chosen. Non-limiting examples of suitable cladding include a metal such as iridium oxide, gold, platinum, or combination of metal films, etc., fluorinated polymer, silicone or other opaque material.

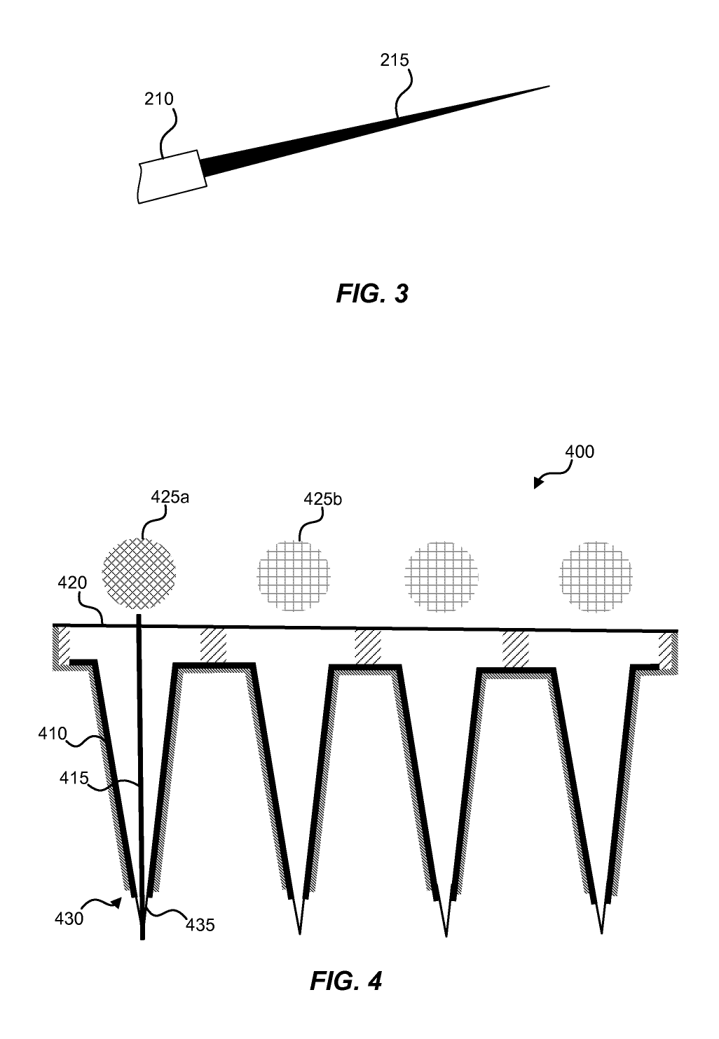

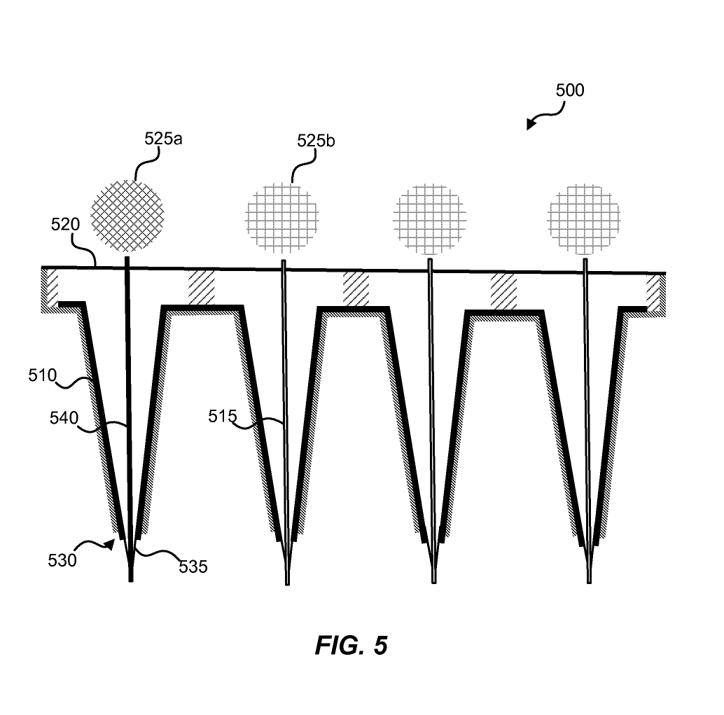

In one optional embodiment, the optical waveguide is a coating along a central shaft of the microelectrode. This optical waveguide has an entrance configured to accept incoming light and direct the light along the optical waveguide to an exit configured to direct the light from the microelectrode into surrounding tissue. FIG. 2 shows several optrodes 210 in a micro-optrode array 200 coated with optical-guide material except at the tips 215 in accordance with an embodiment of the present technology. The coating can have a frustoconical annular shape having an exit which is distanced from the microelectrode tip such that the tip is capable of transmitting electrical impulses to and/or from surrounding tissue. More specifically, the coating can provide a guide for optical signals or optical beams into the tissue. The optrodes can be hollow or solid. In examples where the optrode is solid, the microelectrode tip can be formed of a transparent, electrically conductive material, such as undoped silicon to enable light to pass through the tip and also to enable electrical impulses to be delivered through the tip to biological tissue. FIG. 3 illustrates an expanded view of a single optrode of FIG. 2.

In one aspect, a seamless optical waveguide along the length of the electrodes can be provided by coating each electrode in the array with a thin layer (<5 μm) of optical wave guide material and have an optical-opening close to the electrode tip. Although various methods can be used such as CVD and PVD, CVD allows for more highly conformal coatings to be formed. Further, coatings can be stoichiometrically consistent to improve optical clarity along the waveguide.

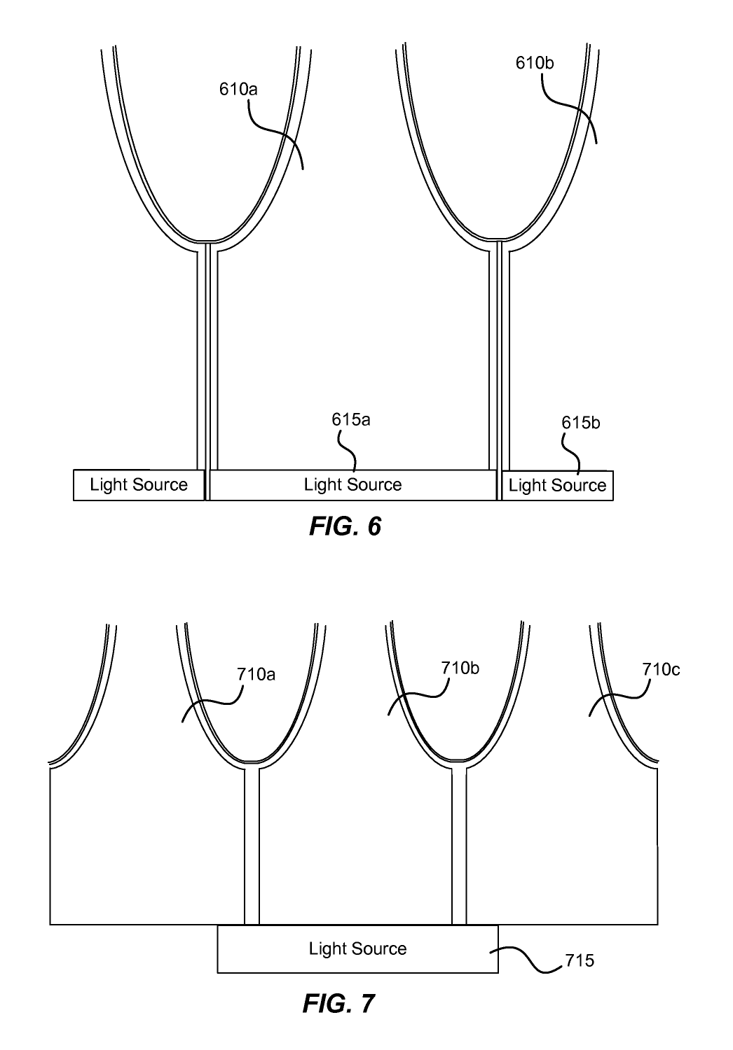

In another optional embodiment, the optical waveguide 415 can be a central shaft of the microelectrode 410 as generally illustrated in FIG. 4. The optical waveguide has an entrance 420 configured to accept incoming light from a light source 425a, 425b and direct the light along the optical waveguide to an exit 430 configured to direct the light from the microelectrode into surrounding tissue. The microelectrode tips 435 can be uncoated such that no cladding material is present so as to allow exit of light from the tips. For example, SIROF coated tips may not emit light. In some cases, the tips can be closed so as to prevent debris or foreign material from entering the electrode if it is hollow. Several options can be suitable to allow light to exit from the tip. For example, 1) the very tip can be masked in advance, or 2) portions of the SIROF removed, leaving a ring of conductive material for electrical recording (or stimulation) that would stick out beyond the parylene insulation, but not cover the very tip. Silicon is highly transparent (nearly 100% transmission for over 1-mm thick silicon) to infrared light from 1200 to 8000 nm wavelength. Therefore the conventional silicon electrodes in the UEA can be used directly as waveguides for near-IR and mid-IR range light.

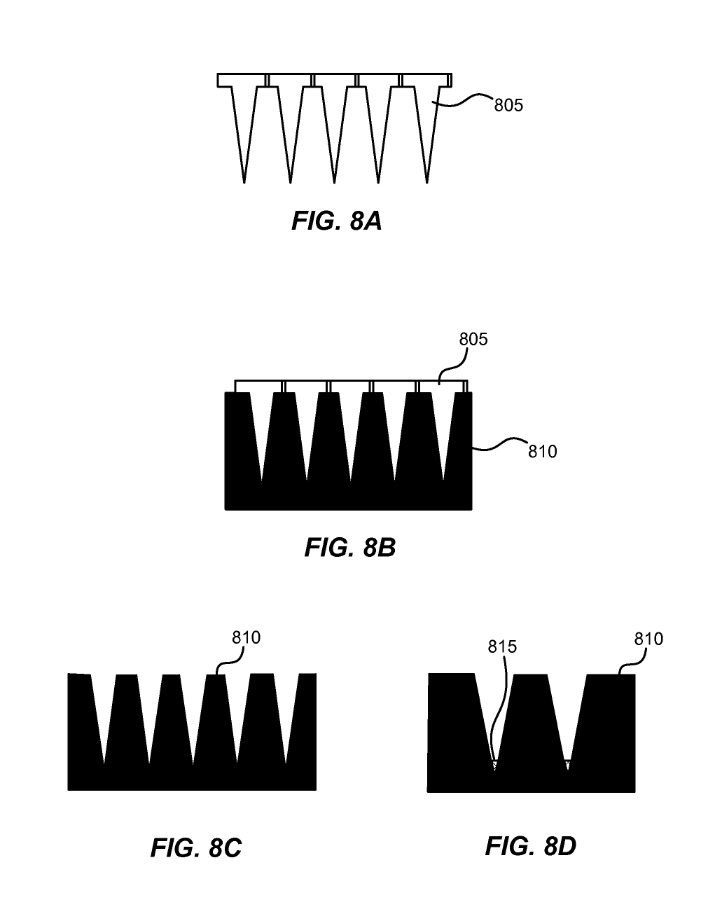

In a third design, microchannels 515 inside the electrodes 510 in an array 500 are used to direct the light 540 from a light source 525a, 525b via an entrance 520 to the electrode tips 535 and out the exit 530 as generally illustrated in FIG. 5. One approach for this design is to create microchannels in conventional, already-fabricated electrodes, using for example Nd:YAG or other micromachining lasers, or Deep Reactive Ion Etching (DRIE), or other processes. Another approach is to create a new type of electrode that is hollow to begin with, using for example a molding and electroplating process based on the USEA as the mold. Either approach for this microchannel technique could be used to transport light of practically any wavelength, including wavelengths not well transmitted through silicon, unlike the second design above. The channels could also be used to deliver agents to neurons to enhance optical stimulation or recording.

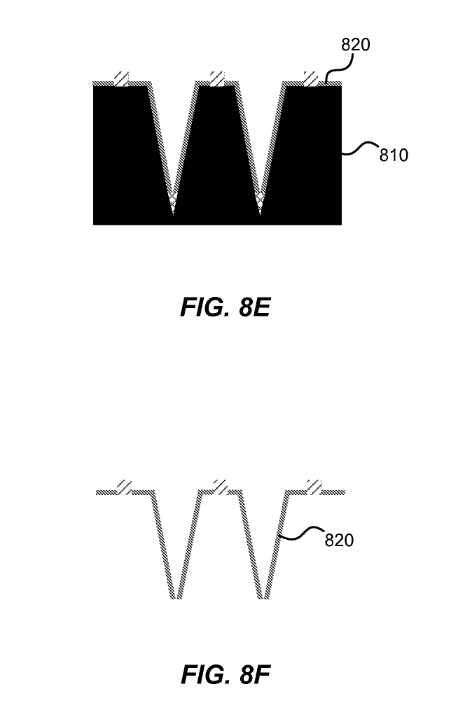

A suitable light source can also be provided and connected to the optical waveguide or waveguides of the interface. A single light source can be used or a plurality of light sources can be used. A single light source can be used to illuminate multiple adjacent optical waveguides simultaneously or light gates can be provided to selectively block light from entering selected waveguides. Optionally, a plurality of light sources can be operatively connected to the optical waveguide of each of the second portion of the plurality of microelectrodes. FIG. 6 illustrates a scenario where each microelectrode 610a, 610b has a corresponding dedicated light source 615a, 615b. In this manner, each of the optical waveguides can be independently addressable. One manufacturing option is to form a grid of trenches (e.g. dicing, etching, etc.) on a substrate backside to a predetermined depth. The individualized surfaces correspond to individual microelectrodes. The trenches can be at least partially filled with a waveguide material which deposits along sidewalls of the trenches. A cladding material can then be filled in to the rest of the trenches to form optical barriers between adjacent waveguide material of adjacent microelectrodes.

Although other options can be used, the light source can be provided by mounting a laser diode (LD) to the backside of the microelectrode at the waveguide inlet. The LD can be preassembled and then mounted to the waveguide inlet or manufactured directly on the backside of the microelectrode. Laser diodes fabricated on the base of individual electrodes will generally transmit more light to the electrode tips than pre-fabricated and assembled diodes because in the former case the reflective losses at the silicon-air/silicon interface are minimal Specialty glass or optically opaque material can be used to electrically and optically isolate the electrodes at the electrode-bottom or base of the array while the electrodes are mechanically held together by the same material.

For optical stimulation, infrared (IR) or other laser source(s) can be either fabricated or assembled on the back of the arrays (e.g., by directly soldering or flip-chip bonding individual diodes or chips to bondpads on the back of the array, placing the light sources on top of the electrode shafts, or via other means). Multiple different sources, controlled independently, would allow stimulation via each electrode independently. Alternatively (in the limit), a single light source can be directed to individual electrodes, e.g., by having controllable microshutters that permit or block light transmission into the hollow microchannels for each electrode.

The optical source can be any suitable light source, although one example is a laser ranging in the near-IR up to 2 μm wavelengths and can be provided as an array of miniaturized (90×90 μm) LDs. For silicon waveguide material, wavelengths from about 1200 nm to 8000 nm can be used. Other wavelengths can also be suitable with corresponding choice of waveguide materials. The array of light sources can be integrated and controlled by an ASIC. An energy input of 10 mJ/cm³ is typically sufficient to stimulate the neurons optically and the thin film silicon oxide waveguides have been shown to have greater than 90% transmittance. Specific energy and transmittance can be optimized for a particular design and choice of materials.

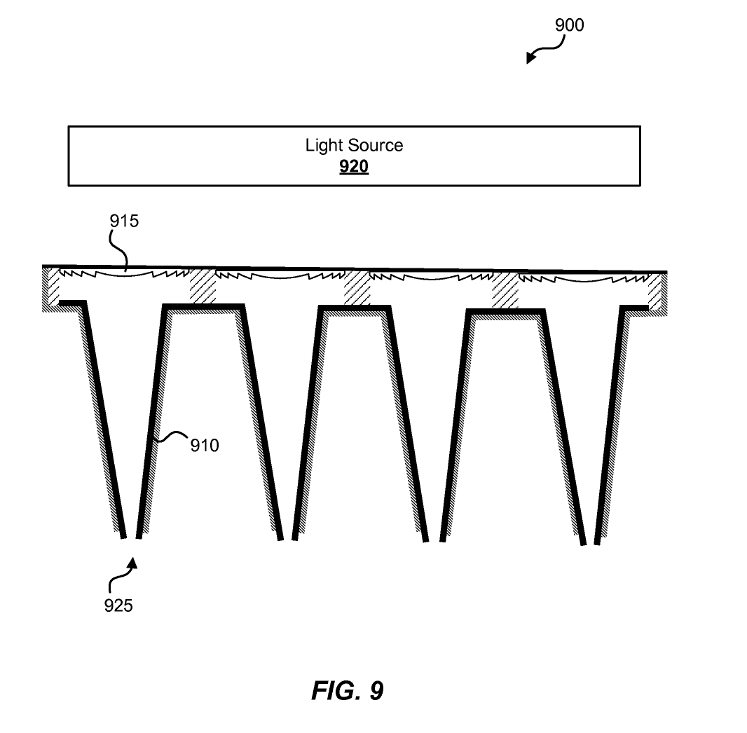

As mentioned above, one option is to provide a base light source oriented in proximity to a localized portion (e.g., 1-9 electrodes) of the second portion of microelectrodes and configured to simultaneously transfer light along multiple adjacent optical waveguides. FIG. 7 illustrates a single light source 715 which is proximate to three waveguide inlets 710a, 710b, 710c. In this case, light will travel through the base along a common path and then split near the base of the tapered electrodes. This approach can simplify manufacturing such that optically opaque material need not be used between microelectrode bases. However, some selectivity will also be lost as a compromise. In some applications, such a compromise will still provide effective stimulation. Because the electrodes are often conventionally separated by glass at the base, the right firing and cooling process of the glass frit will make the glass optically transparent for the light to pass from the source on the other side of the base to the electrode tips through the waveguides. For example, a glass frit can be heated to very high temperatures, some up to 1100° C., for the frit/powder to melt and fuse to form the glass during firing. The optical and mechanical properties of the glass formed by this process can be controlled by the firing temperatures, firing times and the rate of cooling and rate of reaching the firing temperatures. Step temperature profile can optionally be used during ramp-up to and cool-down from firing temperatures to optimize the glass properties.

In a first approach to creating open intraelectrode microchannels, the microchannels can be created in conventional-type, already-fabricated silicon electrodes. For example, Nd:YAG or other micromachining lasers, or DRIE or other processes can be used to create the hollow microchannels by material removal (subtractive process). The tips can optionally be metalized for improved conductivity, e.g., with sputtered iridium. The metallization process can be similar to that used the conventional UEAs/USEAs, except that there is a channel through the tips themselves and their metal coating to emit light. The metal film or other opaque material, e.g. iridium oxide, gold, platinum, or combination of metal films, etc., can be deposited along most of the length of the electrodes to prevent unwanted light escape, while still allowing light to be emitted at the opening of the microchannels at the tip. The metal deposited around the electrode shafts to shield the light can optionally be insulated by an electrically insulating material such as Parylene, except close to the tip area which would act as the electrical recording site.

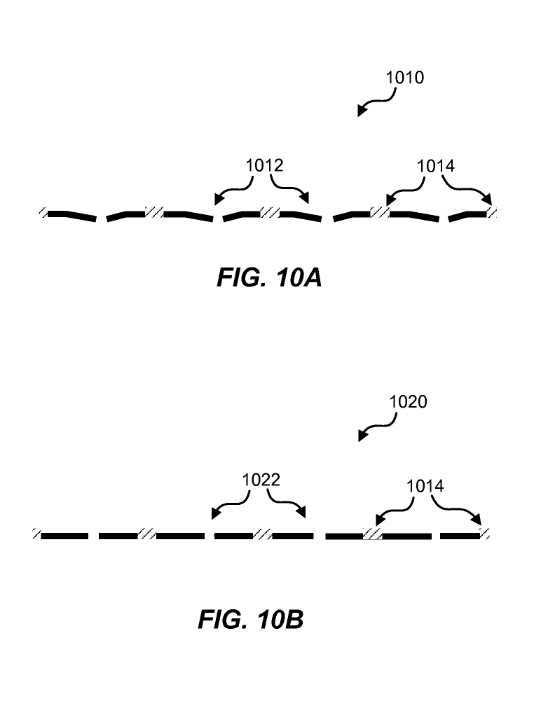

A second approach for creating intraelectrode microchannels is illustrated in FIGS. 8A-8F. A type of electrode can be created that is hollow to begin with (e.g. an additive process). For example, as shown in FIGS. 8A-8F, a molding and electroplating process based on the UEA or USEA as the molded structure. An existing array 805 can be used as a basis for forming the hollow array as in FIG. 8A. The basic idea is to create a mold 810 around an existing array, then remove the mold from the array as in FIG. 8B. The mold can be removed from the array as in FIG. 8C. Subsequently, as shown in FIG. 8D photoresist 815 can be placed in the interior surfaces of the mold to occlude the tip region from subsequent electroplating. As shown in FIG. 8E, the interior of the mold is electroplated with gold 820 or another electrically conductive, light-opaque material (i.e. to act as cladding and as structural walls of the microelectrode). The mold is then removed as in FIG. 8F, leaving behind hollow gold electrodes that are capable transmitting light to the opening in the tip. Because the metal surface is electrically conductive as well as light-opaque, the metal surface can also allow electrical recording of neural signals. Variations of this latter technique can also be used to create supportive, conductive suprastructures around other materials, including light-transmitting materials. In one optional embodiment, the microchannel can have a closed end at the tip which acts a plug to prevent entry of material into the microchannel. The thickness of the plug can be varied in order to minimize light absorption while also providing sufficient structural integrity to block entrance of material into the microchannel.

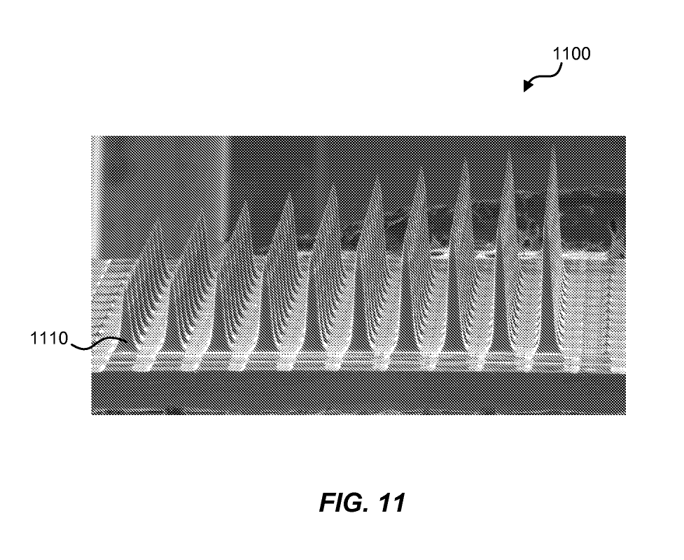

Referring to FIG. 9, an optrode array 900 is shown for a neural interface. The optrode array includes a plurality of optrodes 910. A light source 920 is provided for directing an optical beam into one or more of the optrodes for neural stimulation or activation. The optrode array can also include a Fresnel lens 915. The Fresnel lens can be intrinsically or integrally formed with the optrode array. For example, lithography or other suitable microstructure formation methods may be used to form the Fresnel lenses with the optrode array. Alternately, a Fresnel lens can be separately formed and then positioned to adjacent to an optrode. In other words, the Fresnel lens can be extrinsic and can be placed between the light source and the array. Such a configuration can add additional cost and complexity, such as with alignment issues, but may be desirable for certain applications. With intrinsic Fresnel lenses, alignment issues can be avoided and overall production costs reduced. A Fresnel lens can be formed at or positioned near each of the optrodes and can focus light from the light source toward the tip 925 of the optrode. Focusing of the light can result in a stronger and/or more coherent optical signal transmitted to the biological tissue with which the array may be in contact. While other optical devices may alternately be used in place of the Fresnel lens, the Fresnel lens typically can have a smaller form factor and thus can be more easily integratable with the array.

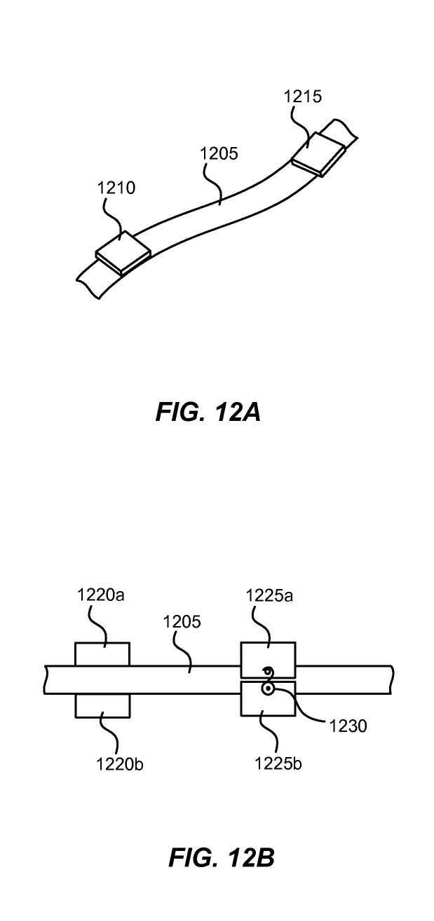

Referring to FIGS. 10A-10B, side cross-section views of optical neural interfaces 1010, 1020 having differing height optrodes are shown in accordance with embodiments of the present technology. Utah optrode arrays (or electrical Utah optrode arrays) can be fabricated with either very short optrodes 1012 separated by an insulating material 1014 (FIG. 10A) or even zero-length optrodes 1022 separated by insulating material 1024 (FIG. 10B). For example, a zero-length optrode array is an array where the optrodes do not extend beyond the flat bottom surface of the Si substrate. Although specific dimensions can vary, non-penetrating optrodes can range in height from about 0 to about 0.5 times a base width. Whereas longer electrodes may be more suitable for intrafasciularly penetrating the nerve, shorter or zero-length electrodes can provide stimulation or activation of neurons extraneurally. Such shorter configurations can utilize a same basic silicon-based approach as used in longer optrode configurations. Many aspects of the technology can be ported directly from the longer electrode approaches, such as: antireflective coatings, coupling of optical fibers from the laser to the array, VCSELs (vertical cavity surface emitting lasers), lenses, and coupling, for example. Extraneural optrode or electrode arrays can be suitable for extraneural stimulation. Such extranueral stimulation may be effective with certain scenarios and within biological limits. Often, nerve stimulation can be more effective when the nerve desired to be activated is in closer proximity to the neural interface. For example, with electrical or optical stimulation of nerves, the nerve desired to be activated is often not on the nerve surface adjacent to the optrode. Heat from the electrical or optical signal can penetrate through to the desired nerve, but in the process the surface tissue or neurons near the surface can be overheated and damaged. Also, as a result, switching times for activating and reactivating the neurons can be increased because there may be more residual heat when heating through to inner neurons from an outer nerve tissue surface. Experimental results of intraneural versus extraneural neural interfaces are described below.

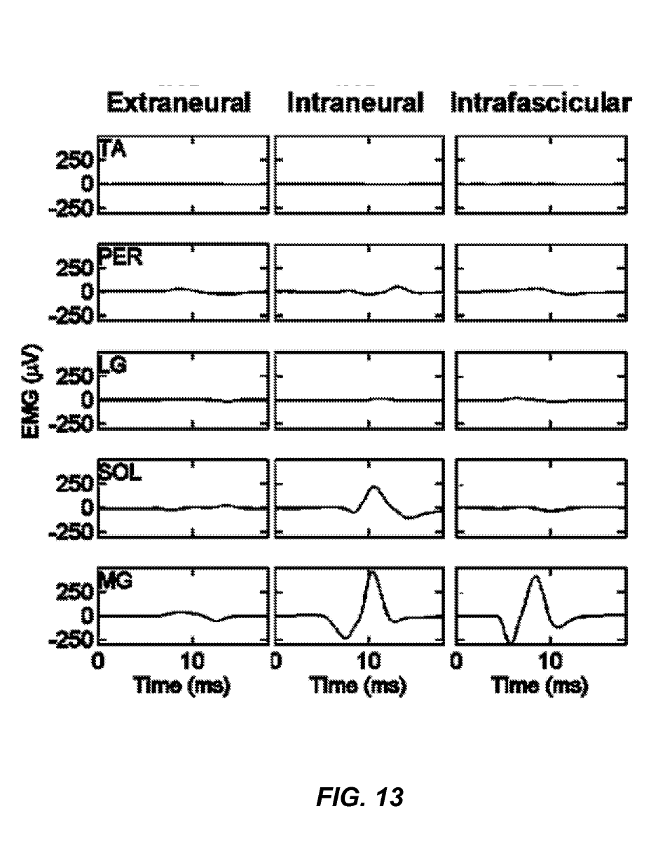

Referring to FIG. 11, an electron microscope image 1100 of a micro-optrode array, wherein rows of micro-optrodes 1110 are formed at different heights in accordance with an embodiment of the present technology. Optrode arrays can be formed where optrodes in a given row are nearly the same length as others in the row, but a different length than optrodes in a different row. Optrodes having different lengths can allow selective activation of fibers at different depths within the nerve. Because optrodes of a same length may activate a same fiber (as also may be the case with extraneural interface heating), use of optrodes of different lengths can allow a higher frequency of safe activation without tissue damage, and by virtue of proximity can activate neurons at different depths from the tissue surface. Such activation could affect either axons (as in nerve, or as in other neural tissue such as cortex), or other neuronal components and processes (e.g., calcium-mediated transmitter release), via either heat or other mechanisms. It will be understood that these representative examples are not exhaustive.

While FIG. 11 illustrates a substantially linear change in optrode height from one row to the next, other configurations are also considered. For example, an array can include rows of optrodes which progressively decrease in height and then progressively increase in height. Such a configuration can form a U or saddle shape when viewed from the side and can be shaped and sized to be implanted on a nerve bundle, such that a center of the nerve bundle is in the middle of the saddle, and the edges of the bundle are contacted by the longer optrodes at the sides. In other words, concave interior surfaces defined by the optrode tips can be formed to better match the curvature of the exterior surface of the nerve. Other variations, such as electrodes of varying heights arranged randomly, alternating height rows of electrodes, arrays where the outer electrodes are longer and inner electrodes (e.g., the electrodes toward the center of the array) are shorter, arrays where the inner electrodes are longer and out are shorter, and so forth, are also contemplated.

In some examples, activation of biological tissue with optrodes can be performed through a trial and error process. More specifically, where the scale of the array and the nerve tissues are small, identifying which optrodes activate which nerves and which optrodes more effectively activate the nerves than other optrodes can be determined by individually testing each of the optrodes. In another example, rather than individually testing each of the optrodes, an electrically conducting portion of the optrode (i.e., the electrical electrode) can be used to measure and record signals from the nerve tissue as the nerves are activated through functioning biological pathways.

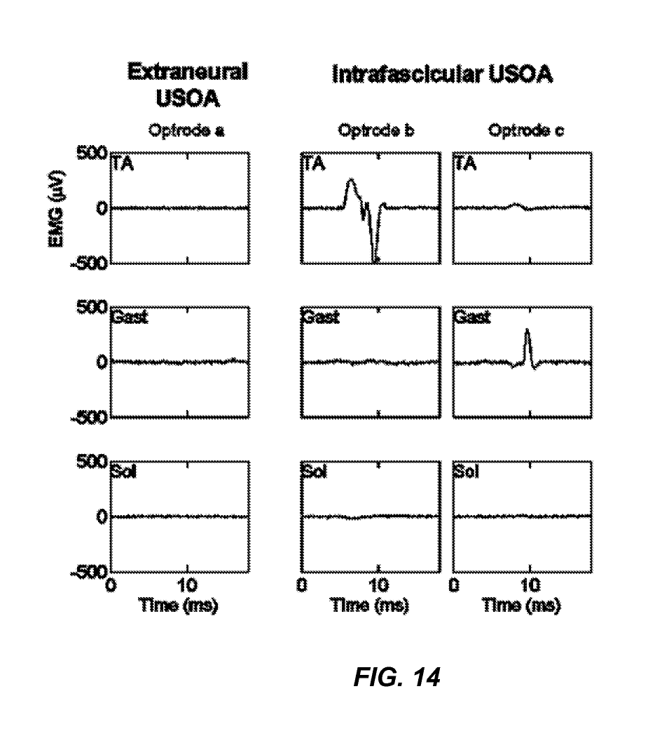

FIGS. 12A-12B illustrate placement of multiple optical neural interfaces on a nerve or nerve bundle in accordance with embodiments of the present technology. Referring to FIG. 12A, use of multiple optrode arrays can increase the number of fibers that can be activated at different stimulations sites. Also, where different arrays are positioned to activate a common fiber, separation of the arrays can enable alternating activation of the same fiber from one array to the next to prevent or minimize overheating and damage of the tissue when a frequency of activation increases (which damage can arise from the cumulative heating effects of infrared light delivered at a single location, as described above). Use of a different array 1215 separated sufficiently from another array 1210 so as to not cause residual heat for the other array, can allow for heat in the tissue 1205 at the different array 1215 to dissipate while the other array 1210 is activated. Even in examples where the different arrays activate different axons, use of multiple, spaced-apart arrays can enable activation of more neurons with less damage than use of larger arrays in a single location.

FIG. 12B illustrates multi-sided optrode array implementations, or rather implementations where optrode arrays are implanted on multiple sides of nerve tissue 1205. Extraneural or intraneural electrode arrays can be manufactured that surround the nerve and stimulate both the top and bottom (and even side) surfaces of the nerve. FIG. 12B illustrates an example where intraneural electrodes 1220a, 1220b are placed on opposite sides of the nerve 1205. Also shown is an example where extraneural electrodes 1225a, 1225b are positioned on opposite sides of the nerve and are latched together using a latch 1230. Use of latching top and bottom electrodes can also enable nerve reshaping (such as has been done with FINEs (Flat Interface Nerve Electrodes)). Some methods, such as nerve twisting can allow two different extraneural arrays to access both superficial and deep surfaces of the nerve, thus addressing at least some of the limitations of extraneural stimulation.

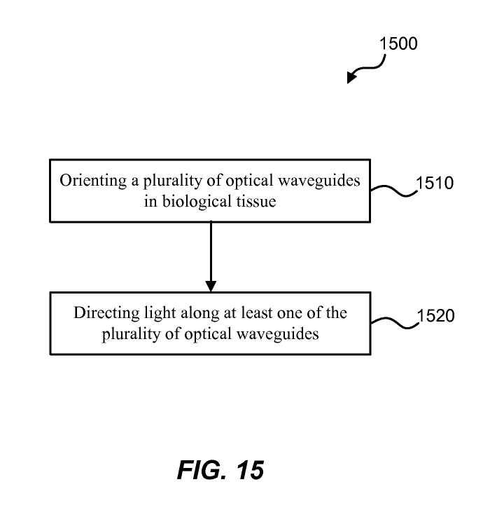

FIGS. 13-14 are graphs showing the performance of various optical neural interfaces in accordance with embodiments of the present technology.

Reference will now be made to FIG. 13. Investigations support the effectiveness of intraneural approaches over extraneural approaches for at least some conditions, as demonstrated in FIG. 13. FIG. 13 illustrates graphs of performance of extraneural, intraneural, and intrafascicular neural interfaces on a sciatic nerve of a cat. Extraneural stimulation (left column) of the posterior side of sciatic nerve produced only a weak response in the medial gastrocnemius (MG) calf muscle. From the multiple locations tested along the nerve length, the MG response was the strongest response obtained. In contrast, intraneural (but extrafascicular) stimulation (second column) evoked a considerably larger response in MG, with some ancillary activity in the adjacent soleus (SOL) calf muscle. Intrafascicular USEA stimulation, at a functionally comparable stimulus strength (third column), produced a comparable but even more selective MG response.

Even in sections of the sciatic nerve that did not respond or responded only weakly to extraneural stimulation (presumably because the underlying fascicles were too far from the surface for axons to be activated), stimulating through the same optrode (fiber optic) intraneurally evoked a strong, reasonably selective response, indicating improved activation capabilities. Further, the shape of the EMG (electromyography) response evoked by intraneural stimulation was nearly identical to that evoked by intrafascicular stimulation with a USEA (Utah Slanted Electrode Array), when USEA stimulus strength (duration) was adjusted to achieve matched response amplitudes. However, USEA intrafascicular stimulation was still more selective (less activation of other muscles) than intraneural stimulation. This experiment used a first-pass, non-optimized methodology, and penetrating the fascicle with the blunt optical probe that was used was not readily possible. Likely, however, intrafascicular stimulation will provide still further benefits relative to intraneural (extrafascicular) stimulation.

Because the efficacy of the various intrafascicular and extrafascicular approaches is demonstrated, electrode- and optrode-based neural interfaces are demonstrated to be able to provide improved sensory feedback in prosthetic limbs. Example uses of such interfaces include: 1) the intrafascicular implanting of Utah Slanted Optrode Arrays (USOAs) in nerves for highly selective stimulation of multiple, independent afferent fibers for sensory feedback; 2) the provision of vertical-cavity surface-emitting laser (VCSEL) packages to provide multiple, independent IR laser sources suitable for integrating with multi-optrode USOAs; and 3) the integration of such devices with a highly advanced neuroprosthetic arm or other type of limb.

In one example, an integrated USOA having 100 optrodes can be formed with a VSCEL array arranged on a side of the substrate opposite from the optrodes. Micro-lenses, such as the previously described Fresnel lenses can be formed or positioned between the optrodes and the VSCEL array. The neural interface can be wired for external control of VCSELs. In another example, the VSCELs are separate and external to the neural interface and the implantation site and optical fibers can be used to carry optical beams from the VSCELs to the optrodes for nerve stimulation.

FIG. 14 includes graphs illustrating comparative effectiveness of extraneural and intrafascicular neural interfaces using the electrode arrays described herein. More specifically, effective and highly selective infrared (IR) optical stimulation was provided with an intrafascicular Utah Slanted Optrode Array (USOA) in a cat sciatic nerve. Myoelectric (EMG) activity was recorded from multiple hind limb muscles, including the tibialis anterior (TA) (an ankle dorsiflexor innervated by the peroneal branch of the sciatic nerve), as well as gastrocnemius (Gast) and soleus (Sol) muscles (ankle plantar flexor muscles innervated by the tibial branch of the sciatic nerve). A 400-um diameter optical fiber connected to a Lockheed Martin Aculight Capella laser source was used to deliver the light to the USOA (wavelength 1873 nm, 5-ms stimulus pulse, ˜0.2-0.4 W at optrode). The optical fiber was positioned via a micromanipulator over the extraneural backplane surface of the USOA. The USOA had 100 individual optrodes of 0.5 to 1.5 mm length on the opposite side from the optical fiber, spaced 400 μm apart in a 10×10 grid configuration. The left column represents extraneural stimulation through individual USOA optrodes (optrode a) resting on the surface of the nerve. These optrodes substantially failed to evoke a response in any instrumented muscle. A representative lack of response is depicted from the TA, Gast, and Sol muscles, though 42 different optrode sites were tested. The center and right columns represent examples where USOA optrodes b and c were implanted intrafascicularly. In contrast with extraneural stimulation, intrafascicular IR stimulation evoked relatively strong and highly selective responses that varied according to which optrode was used for stimulation. Selectivity was exhibited not only between muscles innervated by different branches of the sciatic nerve (e.g., TA vs. Gast and Sol in the center column; or Gast vs. TA in the right column), but also between different muscles innervated by the same branch of the sciatic nerve (e.g., Gast vs. Sol in the right column). These results demonstrate within-branch selectivity at suprathreshold levels for intrafascicular IR stimulation of cat sciatic nerve through USOAs, which has not been previously demonstrated with extraneural IR stimulation of rat sciatic nerve, despite previous intensive investigation. These results indicate that, relative to extraneural IR stimulation, intrafasicular IR stimulation via USOAs can provide a more efficient, highly selective, high-optrode-count device for activating axons in the peripheral nervous system, as well as greater access to fibers in the interior of the nerve.

Relative to other designs for integrating laser stimulation with UEAs/USEAs/UOAs/OSOAs, there would be relatively less loss of light in the microchannels, compared with light absorbed in or reflected from silicon or other waveguide materials. Further, the transmission of light would not depend on its wavelength. For example, certain forms of optical stimulation involving insertion of channelrhodopsin into neurons utilize light wavelengths of 480 nm, a wavelength poorly transmitted by silicon (almost 100% absorbed), and certain voltage sensitive optical reporters also emit light in wavelengths poorly transmitted by silicon.

The microchannels can also be used to administer agents to be incorporated into and modify local neurons, such as light-sensitive molecules for enhanced optical stimulation to excite neurons (e.g., channelrhodopsin) or inhibit neurons (e.g., halorhodopsin), or voltage-dependent, light-emitting molecules (voltage-sensitive optical reporters, e.g. green fluorescent protein (GFP) or voltage-sensitive dyes) for optical recording of neural activity. Similarly, agents can be delivered to enhance responses to light in the infrared portion of the spectrum. Selective promoters or other approaches can be used to allow particular molecules to be incorporated selectively into only certain types of neurons, thereby allowing selective stimulation of only those types of neurons. Note, however, that the use of such agents is not required for this device to be functional. For example, optical stimulation with wavelengths in the infrared or near infrared region can excite neural tissue directly. Such microchannels can also be used to sample the tissue environment.

Notably, portions of these three different approaches can be optionally combined. For example, use of the external waveguides can be combined with electrodes having internal microchannels for delivering agents to be incorporated into and modify local neurons, such as light-sensitive molecules for enhanced optical stimulation, or voltage-dependent, light-emitting molecules for optical recording.

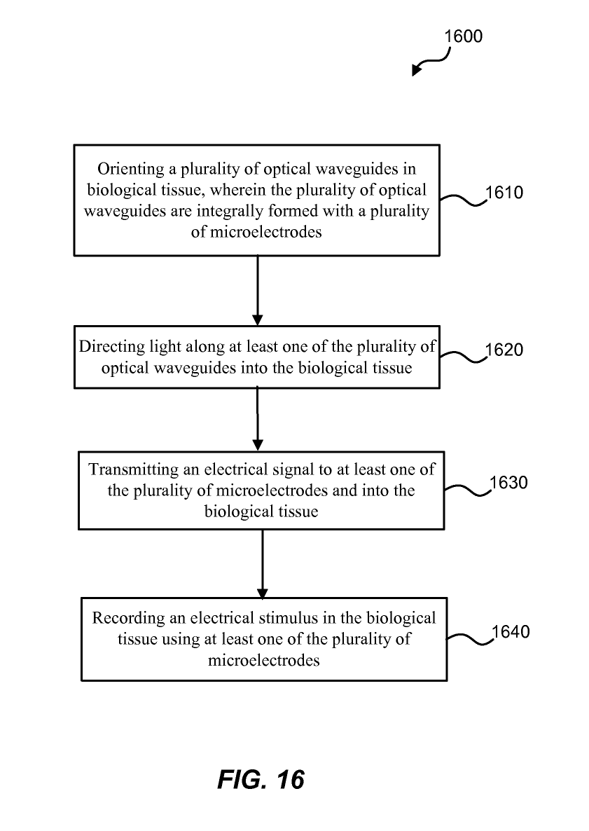

A method 1500 of optically stimulating neurons is shown in FIG. 15 and can include orienting 1510 a plurality of optical waveguides in biological tissue. This can involve placement above a tissue followed by injection of the waveguide into the tissue such that intimate contact of the optical waveguide with neuron-containing tissue is achieved. Light can then be directed 1520 along at least one of the plurality of optical waveguides. Although not always required, the optical waveguides can be oriented along microelectrodes of a microelectrode array such as, but not limited to, those previously mentioned. The method described herein benefits from the limited light penetration through typical tissue. In particular, infrared light penetrates less than about 1 mm into tissue. This limited penetration allows the penetrating optical waveguides to stimulate neurons deep within tissue while also preventing undesired stimulation of neurons which are farther away from the waveguide exit point. As a general guideline, the microelectrode tip can be exposed about 50 μm and the waveguide exit point can be within about 10 μm to about 100 μm of the tip, although other distances can also be suitable.

Another method 1600 of optically stimulating neurons is shown in FIG. 16. The method includes orienting 1610 a plurality of optical waveguides in biological tissue, wherein the plurality of optical waveguides are integrally formed with a plurality of microelectrodes. Light can then be directed 1620 along at least one of the plurality of optical waveguides into the biological tissue. An electrical signal can be transmitted 1630 to at least one of the plurality of microelectrodes and into the biological tissue. An electrical stimulus in the biological tissue can be recorded 1640 using at least one of the plurality of microelectrodes.

This device, besides allowing localized optical stimulation, can sense or record the neuronal electrical activity. In an additional optional feature, the optical waveguides can be used to optically record neural activity. Using IR spectroscopy, the device may be used to optically record the neural activity. Further, optogenetics can be used, i.e. alteration of cells to either convert an optical signal into an electrical impulse or vice versa, conversion of electrical signal into an optical emission that can be recorded using the spectroscopic techniques when the emitted light is transferred from the tip to base of the electrode. Voltage-sensitive dyes can also be injected for optical recording. Electrical neural recording is also possible by fabricating recording sites on the tips of the electrodes, somewhat similar to present conventional microelectrode arrays, but while leaving the tips free to emit light. The electrical recording sites can also serve as electrical stimulation sites. The metal deposited around the electrode shafts to shield the light can be insulated by an electrically insulating material such as Parylene except close to the tip area which would now act as electrical recording site.

This approach integrates the optical stimulation and electrical recording seamlessly into one small device and allows localized selective stimulation, more comprehensive access to nerve fibers, and a wider range of safe stimulation parameters, including higher rates of stimulation suitable for activating neurons in their normal physiological range. This approach also provides penetration into the neural tissue in a way that allows light to be delivered closely to the neurons of interest, and to allow a large number of independent probes. The above methods and devices can also selectively activate subsets of fibers within a single fascicle, and safely use a wide range of stimulation intensities or frequencies, which provides an ability to activate neurons at the full range of physiologically meaningful rates. The present devices also allow a large number of independent stimulation sites and a bidirectional neural interface for stimulating and recording such that a three-dimensional, high-channel count, penetrating optical-electrical neural interface can be provided. As noted above, electrical stimulation capabilities would also be retained through the same pathways used for electrical recording, allowing either mode of stimulation, as well as allowing the comparability and relative advantages of the two forms of stimulation to be compared. In short, this single device provides optical stimulation, electrical stimulation, and electrical recording, and variations of this device also allow for optical recording as described above.

Another advantage of the hybrid optical-electrical device is that the use of optical stimulation can reduce or eliminate stimulation artifacts resulting from conventional electrical stimulation. Such artifacts typically preclude simultaneous electrical recordings from the near vicinity. Hence, it is now possible to maintain recording on adjacent or perhaps even the same electrode through which light stimulation is occurring. Simultaneous recording and stimulation is not readily possible with present wired or wireless UEAs/USEAs. Thus, only a single device can be implanted instead of two devices, which could be advantageous clinically, and which allows new opportunities for investigation of interactions between local neurons.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

What is claimed is:

1. An optical neural interface, comprising:
an optrode array having a plurality of micro-optrodes secured in a common unit such that at least a first portion of the plurality of microelectrodes are independently optically addressable and at least a second portion of the plurality of micro-optrodes include an optical waveguide along the micro-optrodes configured to direct light towards a distal end of the microelectrode.

2. The optical neural interface of claim 1, further comprising a light source configured to emit the light and a lens positioned between the light source and the optical waveguide to direct the light into the optical waveguide.

3. The optical neural interface of claim 2, wherein the lens is a Fresnel lens.

4. The optical neural interface of claim 1, wherein the plurality of micro-optrodes have tips oriented in a common plane.

5. The optical neural interface of claim 1, wherein the plurality of micro-optrodes have tips oriented along a contoured profile.

6. The optical neural interface of claim 1, wherein the plurality of micro-optrodes are non-penetrating.

7. The optical neural interface of claim 1, wherein the optrode array further forms an electrode array wherein the plurality of micro-optrodes are also electrically conductive, and wherein the plurality of micro-optrodes are electrically isolated from one another via an insulating material such that the micro-optrodes are independently addressable.

8. The optical neural interface of claim 1, further comprising a plurality of light sources operatively connected to the optical waveguide of each of the second portion of the plurality of micro-optrodes.

9. The optical neural interface of claim 1, further comprising a base light source oriented in proximity to a localized portion of the second portion of micro-optrode and configured to simultaneously transfer light along multiple adjacent optical waveguides.

10. The optical neural interface of claim 1, wherein the optical waveguide is a coating along a central shaft of the micro-optrode, said optical waveguide having an entrance configured to accept incoming light and direct the light along the optical waveguide to an exit configured to direct the light from the micro-optrode into surrounding tissue.

11. The optical neural interface of claim 10, wherein the coating is a frustoconical annular shape having an exit which is distanced from the micro-optrode tip such that the tip is capable of transmitting optical impulses to and/or from surrounding tissue.

12. The optical neural interface of claim 1, wherein the optical waveguide is a central shaft of the micro-optrode, said optical waveguide having an entrance configured to accept incoming light and direct the light along the optical waveguide to an exit configured to direct the light from the microelectrode into surrounding tissue.

13. The optical neural interface of claim 1, wherein the optical waveguide is a hollow central void within the micro-optrode such that the microelectrode is formed as a needle structure.

14. The optical neural interface of claim 13, wherein the needle structure is configured to allow delivery or sampling of material via the void.

15. The optical neural interface of claim 7, wherein the electrode array is formed of an electrically conductive material such that the micro-optrodes can be used for electrical recording or stimulation.

16. The optical neural interface of claim 1, wherein the optical waveguide is formed of a material selected from the group consisting of silicon, silicon dioxide, silica, acrylic, polystyrene, and combinations thereof.

17. The optical neural interface of claim 1, wherein the optical waveguide further includes cladding to confine light along the optical waveguide, the cladding comprising a metal, fluorinated polymer, or silicon.

18. A method of optically stimulating neurons, comprising:
a) orienting a plurality of optical waveguides in biological tissue; and
b) directing light along at least one of the plurality of optical waveguides.

19. The method of claim 18, wherein said optical waveguides are oriented along microelectrodes of a microelectrode array.

20. The method of claim 19, further comprising electrically addressing the microelectrodes of the microelectrode array independently; and optically addressing the optical waveguides independently.

21. The method of claim 18, further comprising optically recording neural activity using the plurality of optical waveguides.

22. A method of stimulating neurons, comprising:
a) orienting a plurality of optical waveguides in biological tissue, wherein the plurality of optical waveguides are integrally formed with a plurality of microelectrodes;
b) directing light along at least one of the plurality of optical waveguides into the biological tissue;
c) transmitting an electrical signal to at least one of the plurality of microelectrodes and into the biological tissue; and
d) recording an electrical stimulus in the biological tissue using at least one of the plurality of microelectrodes.
